(19) United States
(12) Patent Application Publication
(10) Pub. No.: US 2014/0309516 A1
(43) Pub. Date: Oct. 16, 2014

(54) PHOTOACOUSTIC WAVE MEASUREMENT INSTRUMENT

(71) Applicant: ADVANTEST CORPORATION, Tokyo (JP)

(72) Inventor: Taiichiro IDA, Gunma (JP)

(21) Appl. No.: 14/245,468

(22) Filed: Apr. 4, 2014

(30) Foreign Application Priority Data
Apr. 15, 2013 (JP) 2013-84516

(51) Int. Cl. A61B 5/00

(52) U.S. Cl. CPC A61B 5/0095; A61B 5/489; A61B 5/68
USPC 600/407

(57) ABSTRACT

A photoacoustic wave measurement instrument include a light output unit and at least three photoacoustic wave detection units. The light output unit outputs light. The at least three photoacoustic wave detection units respectively receive photoacoustic waves generated by the light in a measurement object, and convert the photoacoustic wave into electric signals. At least two of the photoacoustic wave detection units have extension directions parallel with or intersecting with each other. At least one of the photoacoustic wave detection units other than the at least two photoacoustic wave detection units has an extension direction intersecting with the extension directions of the at least two photoacoustic wave detection units.

### BACKGROUND ART

1. Field of the Invention

The present invention relates to a position measurement of a target by means of a photoacoustic sensor.

2. Related Art

It is conventionally known to measure a measurement object by detecting photoacoustic waves by using at least two photoacoustic sensors (refer to Patent Document 1). The photoacoustic sensor radiates light upon the measurement object. Then, the light is absorbed by a target in the measurement object. As a result, the target (photoacoustic wave generation part) generates photoacoustic waves. The photoacoustic sensor detects the photoacoustic wave. If the photoacoustic sensor is positioned directly above the target, the photoacoustic sensor can detect the photoacoustic wave. As a result, the position of the target can be measured.

### PRIOR ART DOCUMENTS

(Patent Document 1) Japanese Patent Application Laid-Open (Kokai) No. 2004-201749
(Patent Document 2) Japanese Translation of PCT International Application No. 2011-519281
(Patent Document 3) Japanese Translation of PCT International Application No. 2010-517695
(Patent Document 4) Japanese Laid-Open Patent Publication (Kokai) No. Hei8-320310
(Patent Document 5) Japanese Laid-Open Patent Publication (Kokai) No. 2011-255171

### SUMMARY OF THE INVENTION

However, the photoacoustic wave generated by the target (photoacoustic wave generation part) transmits not only direction directly upward above the target, but also transmits obliquely upward above the target. As a result, if the photoacoustic sensor detects the photoacoustic wave transmitting obliquely upward above the target, the target does not exist directly below the target. In this case, an error is generated in the measurement of the position of the target.

It is therefore an object of the present invention to provide a photoacoustic wave measurement instrument capable of precisely measuring the position of a photoacoustic wave generation part.

According to the present invention, a photoacoustic wave measurement instrument includes: a light output unit that outputs light; and at least three photoacoustic wave detection units that respectively receive photoacoustic waves generated by the light in a measurement object, and convert the photoacoustic wave into electric signals, wherein: at least two of the photoacoustic wave detection units have extension directions parallel with or intersecting with each other; and at least one of the photoacoustic wave detection units other than the at least two photoacoustic wave detection units has an extension direction intersecting with the extension directions of the at least two photoacoustic wave detection units.

According to the thus constructed photoacoustic wave measurement instrument, a light output unit outputs light. At least three photoacoustic wave detection units respectively receive photoacoustic waves generated by the light in a measurement object, and convert the photoacoustic wave into electric signals. At least two of the photoacoustic wave detection units have extension directions parallel with or intersecting with each other. At least one of the photoacoustic wave detection units other than the at least two photoacoustic wave detection units has an extension direction intersecting with the extension directions of the at least two photoacoustic wave detection units.

According to the photoacoustic wave measurement instrument of the present invention, the light output unit may pass inside a polygon constructed by straight lines extending in the extension directions of the photoacoustic wave detection units.

According to the photoacoustic wave measurement instrument of the present invention, the number of photoacoustic wave detection units may be odd.

According to the photoacoustic wave measurement instrument of the present invention, the photoacoustic wave detection units may be arranged at an equal distance from the light output unit.

According to the photoacoustic wave measurement instrument of the present invention, the photoacoustic wave detection units may be arranged at known different distances from the light output unit.

### BRIEF DESCRIPTION OF THE DRAWINGS

FIG. 1 is a cross sectional view of a photoacoustic wave measurement instrument 1 according to a first embodiment of the present invention;

FIG. 2 is a plan view of the photoacoustic wave measurement instrument 1 according to the first embodiment of the present invention;

FIG. 3 is a functional block diagram showing a configuration of the photoacoustic wave measurement device 40 according to the first embodiment of the present invention;

FIG. 4 includes charts showing the relationships between the time and the voltage, which are the measurement results by the electric signal measurement units 41 and 42 of the photoacoustic wave measurement device 40 according to the first embodiment, and shows the measurement result of the electric signals obtained from the photoacoustic wave measurement instrument 1 in FIG. 1(a) (FIG. 4(a)), the measurement result of the electric signals obtained from the photoacoustic wave measurement instrument 1 in FIG. 1(b) (FIG. 4(b)), and the measurement result of the electric signals obtained from the photoacoustic wave measurement instrument 1 in FIG. 1(c) (FIG. 4(c));

FIG. 5 includes a plan view (FIG. 5(a)) of the photoacoustic wave measurement instrument 1 according to the first embodiment of the present invention and a plan view (FIG. 5(b)) of the photoacoustic wave measurement instrument 1 according to the second embodiment of the present invention;

FIG. 6 includes plan views of the photoacoustic wave measurement instrument 1 according to a variation of the second embodiment, and shows a case where the photoacoustic wave detection units 11 and 12 are parallel with each other (FIG. 6(a)) and a case where the photoacoustic wave detection units 11 and 12 intersect with each other (FIG. 6(b));

FIG. 7 includes plan views of the photoacoustic wave measurement instrument 1 according to variations of the second embodiment, and shows a case including six photoacoustic wave detection units 11, 12, 13, 14, 15, and 16 (FIG. 7(a)), and a case including five photoacoustic wave detection units 11, 12, 13, 14, and 15 (FIG. 7(b));

FIG. 8 includes a cross sectional view (FIG. 8(a)) and a plan view (FIG. 8(b)) of the photoacoustic wave measurement instrument 1 according to the third embodiment of the present invention;

FIG. 9 is a cross sectional view of the photoacoustic wave measurement instrument 1 while the photoacoustic wave measurement instrument 1 according to the third embodiment of the present invention is scanned along the measurement object 2;

FIG. 10 includes charts showing relationships between the time and the voltage, which are the measurement results by the electric signal measurement units 41 and 42 of the photoacoustic wave measurement device 40 according to the third embodiment, and shows the measurement result of the electric signals obtained from the photoacoustic wave measurement instrument 1 in FIG. 9(a) (FIG. 10(a)), the measurement result of the electric signals obtained from the photoacoustic wave measurement instrument 1 in FIG. 9(b) (FIG. 10(b)), and the measurement result of the electric signals obtained from the photoacoustic wave measurement instrument 1 in FIG. 9(c) (FIG. 10(c));

FIG. 11 includes a plan view (FIG. 11(a)) of the photoacoustic wave measurement instrument 1 according to the third embodiment of the present invention and a plan view (FIG. 11(b)) of the photoacoustic wave measurement instrument 1 according to the fourth embodiment of the present invention;

FIG. 12 includes plan views of the photoacoustic wave measurement instrument 1 according to a variation of the fourth embodiment, and shows a case where the photoacoustic wave detection units 11 and 12 are parallel with each other (FIG. 12(a)) and a case where the photoacoustic wave detection units 11 and 12 intersect with each other (FIG. 12(b));

FIG. 13 includes plan views of the photoacoustic wave measurement instrument 1 according to variations of the fourth embodiment, and shows a case including six photoacoustic wave detection units 11, 12, 13, 14, 15, and 16 (FIG. 13(a)), and a case including five photoacoustic wave detection units 11, 12, 13, 14, and 15 (FIG. 13(b)); and

FIG. 14 is a functional block diagram showing a configuration of the photoacoustic wave measurement device 40 according to the variation of the first embodiment of the present invention.

### MODE FOR CARRYING OUT THE INVENTION

A description will now be given of an embodiment of the present invention referring to drawings.

### First Embodiment

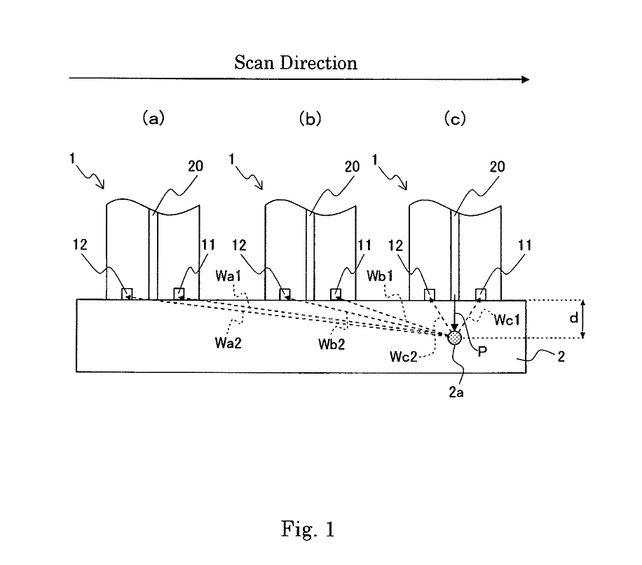

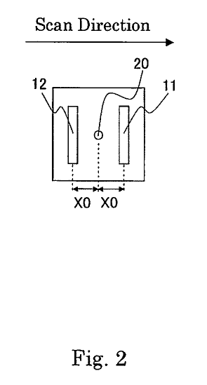

FIG. 1 is a cross sectional view of a photoacoustic wave measurement instrument 1 according to a first embodiment of the present invention. FIG. 2 is a plan view of the photoacoustic wave measurement instrument 1 according to the first embodiment of the present invention. The photoacoustic wave measurement instrument 1 includes photoacoustic wave detection units 11 and 12, and an optical fiber (light output unit) 20. The photoacoustic wave measurement instrument 1 is in contact with a measurement object 2, and is scanned on the measurement object 2 from left to right, for example.

FIG. 1(a) shows the photoacoustic wave measurement instrument 1 positioned far from blood 2a. When the photoacoustic wave measurement instrument 1 shown in FIG. 1(a) is scanned to right, the photoacoustic wave measurement instrument 1 is positioned slightly far from the blood 2a as shown in FIG. 1(b). When the photoacoustic wave measurement instrument 1 shown in FIG. 1(b) is scanned to right, the photoacoustic wave measurement instrument 1 is positioned directly above the blood 2a as shown in FIG. 1(c).

The optical fiber (light output unit) 20 outputs light (such as pulse light P, but continuous light is conceivable). It should be noted that the optical fiber 20 is connected to a pulse light source (not shown) external to the photoacoustic wave measurement instrument 1. The optical fiber 20 passes through the photoacoustic wave measurement instrument 1. Moreover, the pulse light P output from the optical fiber 20 is shown only in FIG. 1(c) for the sake of illustration.

The measurement object 2 is the finger cushion of the human, for example. The blood 2a in a blood vessel exists in the measurement object 2, when the blood 2a in the blood vessel receives the pulse light P, the blood 2a generates photoacoustic waves Wa1 and Wa2 (refer to FIG. 1(a)), photoacoustic waves Wb1 and Wb2 (refer to FIG. 1(b)), and photoacoustic waves Wc1 and Wc2 (refer to FIG. 1(c)).

The photoacoustic wave detection units 11 and 12 receive the photoacoustic waves Wa1, Wa2, Wb1, Wb2, Wc1, and Wc2, and coverts them into electric signals (such as voltages). It is assumed that the photoacoustic wave detection units 11 and 12 are plural. For example, as shown in FIGS. 1 and 2, the number of photoacoustic wave detection units 11 and 12 is two.

Each of the photoacoustic wave detection units 11 and 12 includes a backing material, a piezoelectric element, electrodes, and a spacer which are not shown, and well known. The spacer is in contact with the measurement object 2, the electrodes are placed on the spacer, the piezoelectric element is placed on the electrodes, and the backing material is placed on the piezoelectric element. The photoacoustic waves Wa1, Wa2, Wb1, Wb2, Wc1, and Wc2 are converted into the electric signals (such as voltages) by the piezoelectric element, and extracted to the outside via the electrodes.

Referring to FIG. 2, it should be noted that both the photoacoustic wave detection units 11 and 12 are separated from the optical fiber 20 in a scan direction by a distance X0.

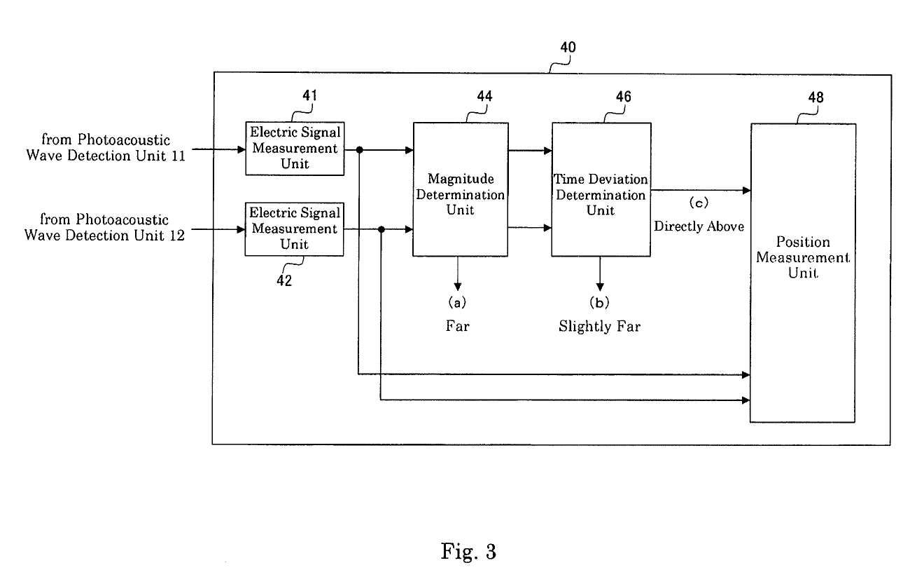

FIG. 3 is a functional block diagram showing a configuration of the photoacoustic wave measurement device 40 according to the first embodiment of the present invention. The photoacoustic wave measurement device 40 includes electric signal measurement units 41 and 42, a magnitude determination unit 44, a time deviation determination unit 46, and a position measurement unit 48. The photoacoustic wave measurement device 40 receives the electric signals from the photoacoustic wave detection units 11 and 12 of the photoacoustic wave measurement instrument 1.

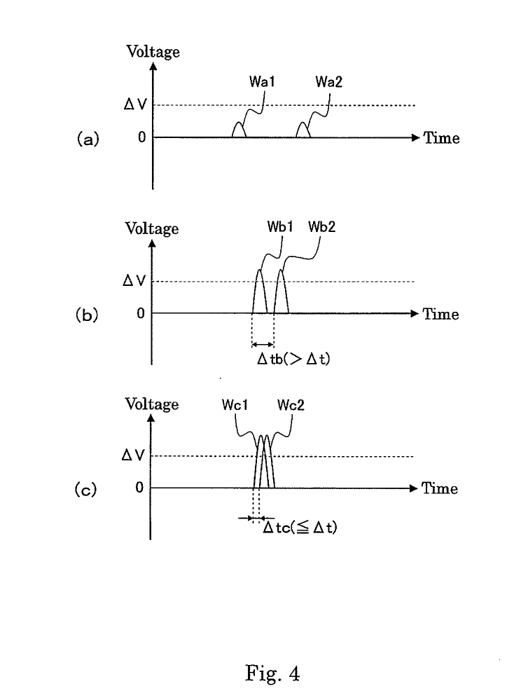

The electric signal measurement unit 41 receives the electric signal from the photoacoustic wave detection unit 11, and outputs a measurement result (such as relationships between the time and the voltage) thereof (refer to Wa1, Wb1, and Wc1 in FIG. 4). The electric signal measurement unit 42 receives the electric signal from the photoacoustic wave detection unit 12, and outputs a measurement result (such as relationships between the time and the voltage) thereof (refer to Wa2, Wb2, and Wc2 in FIG. 4).

The magnitude determination unit 44 receives the measurement results of the electric signals output respectively from the photoacoustic wave detection units 11 and 12 from the electric signal measurement units 41 and 42. Then, the magnitude determination unit 44 determines a magnitude relationship between the magnitude of the electric signal output from each of the photoacoustic wave detection units 11 and 12 and a predetermined threshold ΔV based on the measurement result received from each of the electric signal measurement units 41 and 42.

For example, the magnitude determination unit 44 determines whether both the magnitudes of the electric signals output from the respective photoacoustic wave detection units 11 and 12 are more than (or equal to or more than) the predetermined magnitude threshold ΔV or not.

On this occasion, if the magnitude determination unit 44 determines that both the magnitudes of the electric signals output from the respective photoacoustic wave detection units 11 and 12 are more than the predetermined magnitude threshold ΔV, the magnitude determination unit 44 provides the time deviation determination unit 46 with the measurement results received from the electric signal measurement units 41 and 42.

On the other hand, if the magnitude determination unit 44 determines that at least one of the magnitudes of the electric signals output from the respective photoacoustic wave detection units 11 and 12 is equal to or less than the predetermined magnitude threshold ΔV (refer to FIG. 4(a)), the magnitude determination unit 44 does not provide the time deviation determination unit 46 with the measurement results received from the electric signal measurement units 41 and 42. In this case, the magnitude determination unit 44 may output such a determination result that the photoacoustic wave measurement instrument 1 is positioned far from the blood 2a (refer to FIG. 1(a)).

The time deviation determination unit 46 receives the measurement results via the magnitude determination unit 44 from the electric signal measurement units 41 and 42. Then, the time deviation determination unit 46 determines whether a deviation in time between the electric signals output from the respective photoacoustic wave detection units 11 and 12 is in a predetermined range (equal to or more than 0, and equal to or less than a predetermined time threshold Δt, for example) or not based on the measurement results received from the electric signal measurement units 41 and 42.

For example, the time deviation determination unit 46 determines whether a deviation in time between rising time points of the electric signals output from the respective photoacoustic wave detection units 11 and 12 is equal to or more than 0, and equal to or less than the predetermined time threshold Δt (or equal to or more than 0 and less than Δt) or not.

On this occasion, if the time deviation determination unit 46 determines that a deviation Δtc in time between the rising time points of the electric signals output from the respective photoacoustic wave detection units 11 and 12 is equal to or more than 0, and equal to or less than the predetermined time threshold Δt (refer to FIG. 4(c)), the time deviation determination unit 46 outputs such a determination result that the photoacoustic wave measurement instrument 1 is directly above the blood 2a (refer to FIG. 1(c)) to the position measurement unit 48.

On the other hand, if the time deviation determination unit 46 determines that a deviation Δtb in time between the rising time points of the electric signals output from the respective photoacoustic wave detection units 11 and 12 are more than the predetermined time threshold Δt (refer to FIG. 4(b)), the time deviation determination unit 46 outputs none to the position measurement unit 48. In this case, the time deviation determination unit 46 may output such a determination result that the photoacoustic wave measurement instrument 1 is positioned slightly far from the blood 2a (refer to FIG. 1(b)).

The situation where the time deviation determination unit 46 provides the position measurement unit 48 with such the determination result that the photoacoustic wave measurement instrument 1 is directly above the blood 2a (refer to FIG. 1(c)) means a situation where the magnitude determination unit 44 determines that the magnitudes of the electric signals respectively output from the photoacoustic wave detection units 11 and 12 are more than the threshold ΔV, and simultaneously, the time deviation determination unit 46 determines that the deviation in time between the electric signals respectively output from the photoacoustic wave detection units 11 and 12 is in the predetermined range (equal to or more than 0, and equal to or less than the time threshold Δt).

If the position measurement unit 48 receives such the determination result that the photoacoustic wave measurement instrument 1 is directly above the blood 2a (refer to FIG. 1(c)) from the time deviation determination unit 46, the position measurement unit 48 measures the position of the blood 2a (photoacoustic wave generation part) at which the photoacoustic waves Wc1 and Wc2 are generated in the measurement object 2.

In this case, the position measurement unit 48 measures the position of the blood 2a (photoacoustic wave generation part) while it is assumed that the blood 2a (photoacoustic wave generation part) exists on the extension line of (directly below, for example) the optical fiber (light output unit) 20. The position measurement unit 48 receives the measurement results from the electric signal measurement units 41 and 42, thereby measuring the position of the blood 2a (photoacoustic wave generation part). For example, a depth d of the blood 2a with respect to a surface of the measurement object 2 may be measured. It is assumed that it is found out that a time taken by the photoacoustic wave Wc1 to reach the photoacoustic wave detection unit 11 from the blood 2a and a time taken by the photoacoustic wave Wc2 to reach the photoacoustic wave detection unit 12 from the blood 2a are both T based on the measurement results received from the electric signal measurement units 41 and 42. Then, (T×Vs)²=d²+X0² holds true, where Vs is a velocity of the photoacoustic wave in the measurement object 2. X0 and Vs are known, and the depth d of the blood 2a can thus be obtained.

A description will now be given of an operation of the first embodiment of the present invention.

First, before the description of the operation, the positional relationships between the photoacoustic wave measurement instrument 1 and the blood 2a in FIGS. 1(a), 1(b), and 1(c), and the relationships between the results of the comparison of the electric signal with the magnitude threshold ΔV and the time threshold Δt are shown in Table 1.

TABLE 1

| | | | EQUAL TO OR |
|---|---|---|---|
| | | MORE THAN | LESS THAN TIME |
| | DISTANCE FROM | MAGNITUDE | DEVIATION |
| | BLOOD 2a | THRESHOLD ΔV | THRESHOLD Δt |
| (a) | FAR | x | — |
| (b) | SLIGHTLY FAR | ∘ | x |
| (c) | DIRECTLY | ∘ | ∘ |
| | ABOVE | | |

∘: Condition is satisfied
x: Condition is not satisfied
—: Not determined

When the scan of the photoacoustic wave measurement instrument 1 starts, the photoacoustic wave measurement instrument 1 is positioned far from the blood 2a as shown in FIG. 1(a).

On this occasion, the external pulse light source (not shown) generates the pulse light P, and the pulse light P is output from the optical fiber 20. The pulse light P is fed to the measurement object 2.

The pulse light P reaches the blood 2a in the blood vessel of the measurement object 2. Then, the blood 2a in the blood vessel absorbs the pulse light P, and the dilatational waves (photoacoustic waves Wa1 and Wa2) are output from the blood 2a in the blood vessel.

The photoacoustic waves Wa1 and Wa2 transmit through the measurement object 2, and reach the photoacoustic wave detection units 11 and 12. The photoacoustic wave detection units 11 and 12 respectively convert pressures of the photoacoustic waves Wa1 and Wa2 into the electric signals (such as voltages). The voltages are fed to the electric signal measurement units 41 and 42 of the photoacoustic wave measurement device 40.

FIG. 4 includes charts showing the relationships between the time and the voltage, which are the measurement results by the electric signal measurement units 41 and 42 of the photoacoustic wave measurement device 40 according to the first embodiment, and shows the measurement result of the electric signals obtained from the photoacoustic wave measurement instrument 1 in FIG. 1(a) (FIG. 4(a)), the measurement result of the electric signals obtained from the photoacoustic wave measurement instrument 1 in FIG. 1(b) (FIG. 4(b)), and the measurement result of the electric signals obtained from the photoacoustic wave measurement instrument 1 in FIG. 1(c) (FIG. 4(c)).

(a) When Photoacoustic Wave Measurement Instrument 1 is Far from Blood 2a

As shown in FIG. 1(a), the photoacoustic wave measurement instrument 1 is far from the blood 2a. Thus, the photoacoustic waves Wa1 and Wa2 are weak, and the magnitudes of the electric signals (voltages) obtained from the photoacoustic waves Wa1 and Wa2 are small, and are both equal to or less than the magnitude threshold ΔV (refer to FIG. 4(a)).

In this case, the magnitude determination unit 44 does not feed the measurement results received from the electric signal measurement units 41 and 42 to the time deviation determination unit 46. The magnitude determination unit 44 outputs such the determination result that the photoacoustic wave measurement instrument 1 is positioned far from the blood 2a (refer to FIG. 1(a)).

(b) When Photoacoustic Wave Measurement Instrument 1 is Slightly Far from Blood 2a

When the photoacoustic wave measurement instrument 1 is scanned from the state shown in FIG. 1(a), the photoacoustic wave measurement instrument 1 is positioned slightly far from the blood 2a as shown in FIG. 1(b).

As shown in FIG. 1(b), though the photoacoustic wave measurement instrument 1 is slightly far from the blood 2a, the photoacoustic wave measurement instrument 1 is closer to the blood 2a than the photoacoustic wave measurement instrument 1 in the state shown in FIG. 1(a). Thus, the photoacoustic waves Wb1 and Wb2 are stronger than the photoacoustic waves Wa1 and Wa2, and both the magnitudes of the electric signals (voltages) obtained from the photoacoustic waves Wb1 and Wb2 are more than the magnitude threshold ΔV (refer to FIG. 4(b)).

In this case, the magnitude determination unit 44 feeds the measurement results received from the electric signal measurement units 41 and 42 to the time deviation determination unit 46.

As shown in FIG. 1(b), the photoacoustic wave measurement instrument 1 is slightly far from the blood 2a, and the difference between a distance traveled by the photoacoustic wave Wb1 and a distance traveled by the photoacoustic wave Wb2 is thus not negligible. Thus, a difference between the time taken by the photoacoustic wave Wb1 to reach the photoacoustic wave detection unit 11 and the time taken by the photoacoustic wave Wb2 to reach the photoacoustic wave detection unit 12 is not negligible either. Therefore, the deviation Δtb in time between the rising time points of the electric signals obtained from the photoacoustic waves Wb1 and Wb2 is not negligible (for example, more than the predetermined time threshold Δt) referring to FIG. 4(b).

In this case, the time deviation determination unit 46 does not specifically output anything to the position measurement unit 48. The time deviation determination unit 46 outputs such the determination result that the photoacoustic wave measurement instrument 1 is positioned slightly far from the blood 2a (refer to FIG. 1(b)).

(c) When Photoacoustic Wave Measurement Instrument 1 is Directly Above Blood 2a

When the photoacoustic wave measurement instrument 1 is scanned from the state shown in FIG. 1(b), the photoacoustic wave measurement instrument 1 is positioned directly above the blood 2a as shown in FIG. 1(c).

As shown in FIG. 1(c), the photoacoustic wave measurement instrument 1 is closer to the blood 2a than the photoacoustic wave measurement instrument 1 in the state shown in FIG. 1(a). Thus, the photoacoustic waves Wc1 and Wc2 are stronger than the photoacoustic waves Wa1 and Wa2, and both the magnitudes of the electric signals (voltages) obtained from the photoacoustic waves Wc1 and Wc2 are more than the magnitude threshold ΔV (refer to FIG. 4(c)).

In this case, the magnitude determination unit 44 feeds the measurement results received from the electric signal measurement units 41 and 42 to the time deviation determination unit 46.

As shown in FIG. 1(c), the photoacoustic wave measurement instrument 1 is directly above the blood 2a. Moreover, referring to FIG. 2, the distance between the photoacoustic wave detection unit 11 and the optical fiber 20 and the distance between the photoacoustic wave detection unit 12 and the optical fiber 20 are both X0, and are equal to each other. Thus, the distance traveled by the photoacoustic wave Wc1 and the distance traveled by the photoacoustic wave Wc2 are equal to each other. Thus, the time taken by the photoacoustic wave Wc1 to reach the photoacoustic wave detection unit 11 and the time taken by the photoacoustic wave Wc2 to reach the photoacoustic wave detection unit 12 are equal to each other. Therefore, the deviation Δtc in time between the rising time points of the electric signals obtained from the photoacoustic waves Wc1 and Wc2 is so small as to be negligible (for example, equal to or less than the predetermined time threshold Δt) referring to FIG. 4(c).

In this case, the time deviation determination unit 46 outputs such the determination result that the photoacoustic wave measurement instrument 1 is positioned directly above the blood 2a (refer to FIG. 1(c)) to the position measurement unit 48. The position measurement unit 48 measures the position of the blood 2a (photoacoustic wave generation part) while it is assumed that the blood 2a (photoacoustic wave generation part) exists on the extension line of (directly below, for example) the optical fiber 20. The position measurement unit 48 receives the measurement results from the electric signal measurement units 41 and 42, thereby measuring the position (such as the depth d of the blood 2a) of the blood 2a (photoacoustic wave generation part).

It is possible to determine whether the blood 2a (photoacoustic wave generation part) exists on the extension line of (directly below, for example) the optical fiber 20 of the photoacoustic wave measurement instrument 1 (refer to FIG. 1(c)) or not (refer to FIGS. 1(a) and (b)) according to the first embodiment.

Moreover, the position measurement unit 48 measures the position of the blood 2a while it is assumed that the blood 2a exists on the extension line of the optical fiber 20 of the photoacoustic wave measurement instrument 1 in the photoacoustic wave measurement device 40. On this occasion, the photoacoustic wave measurement device 40 carries out such the measurement when the blood 2a actually exists on the extension line of the optical fiber 20 of the photoacoustic wave measurement instrument 1 (refer to FIG. 1(c)). The position of the blood 2a thus can be precisely measured by the photoacoustic wave measurement instrument 1.

A description is given of the first embodiment while it is assumed that the photoacoustic wave measurement device 40 includes the magnitude determination unit 44. However, such a variation that the photoacoustic wave measurement device 40 does not include the magnitude determination unit 44 is conceivable.

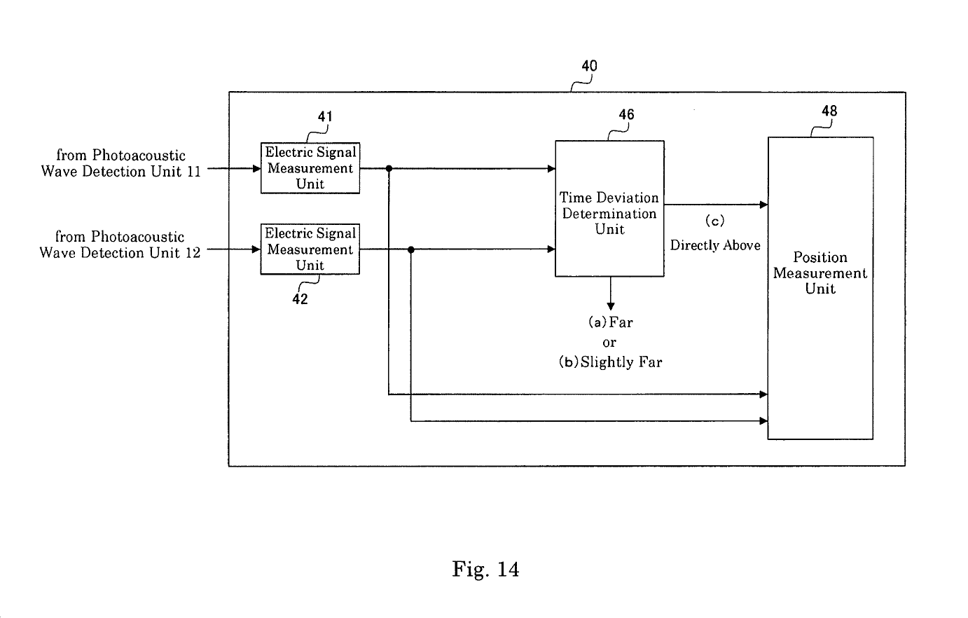

FIG. 14 is a functional block diagram showing a configuration of the photoacoustic wave measurement device 40 according to the variation of the first embodiment of the present invention. The photoacoustic wave measurement device 40 according to the variation of the first embodiment of the present invention includes the electric signal measurement units 41 and 42, the time deviation determination unit 46, and the position measurement unit 48.

The electric signal measurement units 41 and 42 and the position measurement unit 48 are the same as those of the first embodiment (refer to FIG. 3), and hence a description thereof is omitted.

The time deviation determination unit 46 directly (not via the magnitude determination unit 44) receives the measurement results from the electric signal measurement units 41 and 42. The determination method by the time deviation determination unit 46 is the same as that of the first embodiment, and a description thereof, therefore, is omitted.

However, if the time deviation determination unit 46 determines that the time deviation Δtb in time between the rising time points of the electric signals respectively output from the photoacoustic wave detection units 11 and 12 is more than the predetermined time threshold Δt, the time deviation determination unit 46 outputs such a determination result that the photoacoustic wave measurement instrument 1 is positioned far from (refer to FIG. 1(a)), or slightly far from (refer to FIG. 1(b)) the blood 2a. If the photoacoustic wave measurement instrument 1 is positioned slightly far from the blood 2a (refer to FIG. 1(b)), Δtb is more than Δt, and if the photoacoustic wave measurement instrument 1 is positioned far from the blood 2a (refer to FIG. 2(a)), Δtb further increases, and Δtb further exceeds Δt.

If the photoacoustic wave measurement instrument 1 is positioned far from the blood 2a (refer to FIG. 1(a)), the photoacoustic waves Wa1 and Wa2 are weak. However, if the electric signal measurement units 41 and 42 can carry out precise measurement high in S/N ratio, even if the photoacoustic wave measurement instrument 1 is positioned far from the blood 2a, the deviation in time between the rising time points of the electric signals respectively output from the photoacoustic wave detection units 11 and 12 can be measured, and the time deviation determination unit 46 can makes the determination.

The photoacoustic wave measurement device 40 according to the variation of the first embodiment can provide the same effect as of the first embodiment. It should be noted that a measurement by such a variation that the photoacoustic wave measurement device 40 does not include the magnitude determination unit 44 can be made in other embodiments.

### Second Embodiment

The photoacoustic wave measurement instrument 1 according to a second embodiment is different from the photoacoustic wave measurement instrument 1 according to the first embodiment in that the photoacoustic wave measurement instrument 1 according to the second embodiment includes at least three photoacoustic wave detection units.

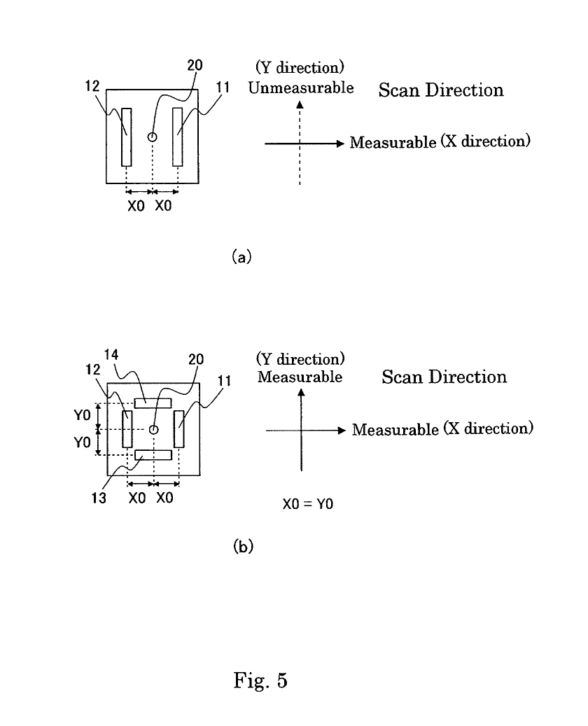

FIG. 5 includes a plan view (FIG. 5(a)) of the photoacoustic wave measurement instrument 1 according to the first embodiment of the present invention and a plan view (FIG. 5(b)) of the photoacoustic wave measurement instrument 1 according to the second embodiment of the present invention.

The photoacoustic wave measurement instrument 1 according to the second embodiment of the present invention includes photoacoustic wave detection units 11, 12, 13, and 14, and the optical fiber (light output unit) 20. In the following section, like components are denoted by like numerals as of the first embodiment, and will be described in no more details.

Referring to FIG. 5(b), the photoacoustic wave measurement instrument 1 according to the second embodiment is in contact with the measurement object 2, and carries out the scan above the measurement object 2 from the left to the light (in an X direction) or scans from the rear to the front (in a Y direction, for example). In other words, the scan can be carried out in the two directions orthogonal to each other. It should be noted that the scan can be carried out at the same time in the two directions orthogonal to each other (for example, from the obliquely left rear to the obliquely right front).

The photoacoustic wave detection units 11 and 12 and the optical fiber (light output unit) 20 are the same as those of the first embodiment, and a description thereof, therefore, is omitted.

It should be noted that, referring to FIG. 5(b), the photoacoustic wave measurement instrument 1 according to the second embodiment has the four photoacoustic wave detection units.

Configurations of the photoacoustic wave detection units 13 and 14 are the same as the configurations of the photoacoustic wave detection units 11 and 12. It should be noted that both the photoacoustic wave detection units 13 and 14 are separated by a distance Y0 in the Y direction from the optical fiber 20. A relationship Y0=X0 may hold true, and the photoacoustic wave detection units 11, 12, 13, and 14 are arranged at the equal distance X0 (=Y0) from the optical fiber 20 in this case.

It should be noted that the photoacoustic wave detection units 11 and 12 extend in the direction (Y direction) orthogonal to the direction of the scan (X direction). Moreover, it should be noted that the photoacoustic wave detection units 13 and 14 extend in the direction (X direction) orthogonal to the direction of the scan (Y direction). Thus, the extension direction (Y direction) of the photoacoustic wave detection unit 11 and the extension direction (Y direction) of the photoacoustic wave detection unit 12 are parallel with each other, and the extension direction (X direction) of the photoacoustic wave detection unit 13 and the extension direction (X direction) of the photoacoustic wave detection unit 14 intersect with the extension direction (Y direction) of the photoacoustic wave detection unit 11 and the extension direction (Y direction) of the photoacoustic wave detection unit 12.

The configuration of the photoacoustic wave measurement device 40 according to the second embodiment of the present invention is the same as that of the first embodiment (refer to FIG. 3), and hence an illustration and a description thereof are omitted. It should be noted that the electric signal measurement units are provided as many as the number (four) of the photoacoustic wave detection units.

A description will now be given of an operation of the second embodiment of the present invention while comparing the second embodiment with the first embodiment.

The photoacoustic wave measurement instrument 1 according to the first embodiment of the present invention shown in FIG. 5(a) can measure the measurement object 2 while the scan is carried out in the X direction so as to pass directly above the blood 2a as described before. However, the distance traveled by the photoacoustic wave received by the photoacoustic wave detection unit 11 and the distance traveled by the photoacoustic wave received by the photoacoustic wave detection unit 12 are the same regardless of the distance of the photoacoustic wave measurement instrument 1 from the blood 2a, and the deviation in time between the time points at which the electric signals obtained from the respective photoacoustic waves rise is thus 0. Therefore, if the scan is carried out in the Y direction so as to pass directly above the blood 2a, the measurement object 2 cannot be measured.

However, the distance traveled by the photoacoustic wave received by the photoacoustic wave detection unit 13 and the distance traveled by the photoacoustic wave received by the photoacoustic wave detection unit 14 are different from each other depending on the distance of the photoacoustic wave measurement instrument 1 from the blood 2a, and the deviation in time between the time points at which the electric signals obtained from the respective photoacoustic waves rise thus changes. Therefore, the photoacoustic wave measurement instrument 1 according to the second embodiment of the present invention shown in FIG. 5(b) can measure the measurement object 2 even if the scan is carried out in the Y direction so as to pass directly above the blood 2a.

The distance traveled by the photoacoustic wave received by the photoacoustic wave detection unit 11 and the distance traveled by the photoacoustic wave received by the photoacoustic wave detection unit 12 are different from each other depending on the distance of the photoacoustic wave measurement instrument 1 from the blood 2a (refer to the first embodiment), and the deviation in time between the time points at which the electric signals obtained from the respective photoacoustic waves rise thus changes. Therefore, the photoacoustic wave measurement instrument 1 according to the second embodiment of the present invention shown in FIG. 5(b) can measure the measurement object 2 while the scan is carried out in the X direction so as to pass directly above the blood 2a.

The photoacoustic wave measurement apparatus 40 according to the second embodiment converts the photoacoustic waves obtained from the photoacoustic wave detection units 11, 12, 13, and 14 into the electric signals by the four electric signal measurement units provided as many as the number of the photoacoustic wave detection units. Then, the magnitude determination unit 44 determines whether each of the magnitudes (voltages) of the electric signals is more than the magnitude threshold ΔV or not. Then, the time deviation determination unit 46 determines whether the deviation in time between the time points at which each of pairs of electric signals rise is equal to or less than the predetermined time threshold Δt or not. As a result, the measurement object 2 can be measured.

The photoacoustic wave measurement instrument 1 according to the second embodiment can carry out the scan above the measurement object 2 in the two directions orthogonal to each other, for example from the left to the right (in the X direction), or from the rear to the front (in the Y direction). It should be noted that the scan can be carried out at the same time in the two directions orthogonal to each other (for example, from the obliquely left rear to the obliquely right front).

It should be noted that variations of the photoacoustic wave measurement instrument 1 according to the second embodiment of the present invention are conceivable.

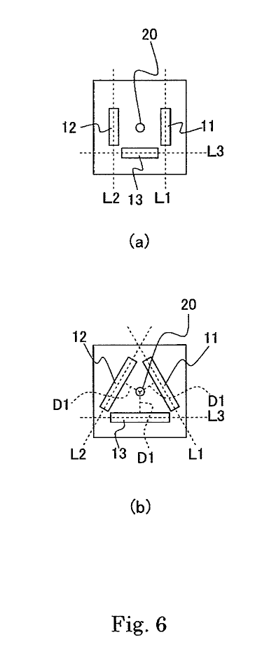

FIG. 6 includes plan views of the photoacoustic wave measurement instrument 1 according to a variation of the second embodiment, and shows a case where the photoacoustic wave detection units 11 and 12 are parallel with each other (FIG. 6(a)) and a case where the photoacoustic wave detection units 11 and 12 intersect with each other (FIG. 6(b)).

The photoacoustic wave measurement instrument 1 includes the three photoacoustic wave detection units 11, 12, and 13 in the variation of the second embodiment shown in FIG. 6.

FIG. 6(a) is the plan view of the photoacoustic wave measurement instrument 1 according to the variation where the photoacoustic wave detection units 11 and 12 are parallel with each other. The variation shown in FIG. 6(a) is in a shape where the photoacoustic wave detection unit 14 is absent in the photoacoustic wave measurement instrument 1 (refer to FIG. 5(b)) according to the second embodiment.

Extension directions L1 and L2 of at least two photoacoustic wave detection units 11 and 12 out of the photoacoustic wave detection units 11, 12, and 13 are parallel with each other. An extension direction L3 of the at least one photoacoustic wave detection unit 13 other than the two photoacoustic wave detection units intersects with the extension directions L1 and L2 of the two photoacoustic wave detection units 11 and 12.

The distances traveled by the photoacoustic waves received by the photoacoustic wave detection units 11 and 12 and the distance traveled by the photoacoustic wave received by the photoacoustic wave detection unit 13 are different from each other depending on the distance of the photoacoustic wave measurement instrument 1 from the blood 2a, and the deviation in time between the time points at which the electric signals obtained from the respective photoacoustic waves rise thus changes. Therefore, according to the variation shown in FIG. 6(a), even if the scan is carried out in the Y direction so as to pass directly above the blood 2a, the measurement object 2 can be measured.

Therefore, according to the variation shown in FIG. 6(a), even if the scan is carried out in the X direction so as to pass directly above the blood 2a, the measurement object 2 can be measured. The distance traveled by the photoacoustic wave received by the photoacoustic wave detection unit 11 and the distance traveled by the photoacoustic wave received by the photoacoustic wave detection unit 12 are different from each other depending on the distance of the photoacoustic wave measurement instrument 1 from the blood 2a (refer to the first embodiment), and the deviation in time between the time points at which the electric signals obtained from the respective photoacoustic waves rise thus changes.

FIG. 6(b) is the plan view of the photoacoustic wave measurement instrument 1 according to the variation where the photoacoustic wave detection units 11 and 12 intersect with each other.

Extension directions L1 and L2 of at least two photoacoustic wave detection units 11 and 12 out of the photoacoustic wave detection units 11, 12, and 13 intersect with each other. An extension direction L3 of the at least one photoacoustic wave detection unit 13 other than the two photoacoustic wave detection units intersects with the extension directions L1 and L2 of the two photoacoustic wave detection units 11 and 12.

It should be noted that the light output unit 20 passes through an inside of a polygon (a regular triangle in FIG. 6(b)) constructed by straight lines extending in the extension directions L1, L2, and L3 in the variation shown in FIG. 6(b). Moreover, all distances between the light output unit 20 and the photoacoustic wave detection units 11, 12, and 13 are equal to D1.

According to the variation shown in FIG. 6(b), even if the scan is carried out in the Y direction so as to pass directly above the blood 2a, the measurement object 2 can be measured. The distance traveled by the photoacoustic wave received by the photoacoustic wave detection unit 11 and the distance traveled by the photoacoustic wave received by the photoacoustic wave detection unit 12 are the same regardless of the distance of the photoacoustic wave measurement instrument 1 from the blood 2a. However, the distances traveled by the photoacoustic waves received by the photoacoustic wave detection units 11 and 12 and the distance traveled by the photoacoustic wave received by the photoacoustic wave detection unit 13 are different from each other depending on the distance of the photoacoustic wave measurement instrument 1 from the blood 2a, and the deviation in time between the time points at which the electric signals obtained from the respective photoacoustic waves rise thus changes. Thus, according to the variation shown in FIG. 6(b), even if the scan is carried out in the Y direction so as to pass directly above the blood 2a, the measurement object 2 can be measured.

The distance traveled by the photoacoustic wave received by the photoacoustic wave detection unit 11 and the distance traveled by the photoacoustic wave received by the photoacoustic wave detection unit 12 are different from each other depending on the distance of the photoacoustic wave measurement instrument 1 from the blood 2a, and the deviation in time between the time points at which the electric signals obtained from the respective photoacoustic waves rise thus changes. Therefore, according to the variation shown in FIG. 6(a), even if the scan is carried out in the X direction so as to pass directly above the blood 2a, the measurement object 2 can be measured.

It should be noted that the photoacoustic wave detection units 11 and 12 are parallel with each other (refer to FIG. 6(a)) or intersect with each other (refer to FIG. 6(b)) in the variation of the second embodiment, and the photoacoustic wave detection unit 12 is never disposed on the extension of the photoacoustic wave detection unit 11.

Whether the photoacoustic wave detection units 11 and 12 are parallel with each other, or intersect with each other as shown in FIG. 6, if there is the one photoacoustic wave detection unit 13 extending so as to intersect with the extension directions of the photoacoustic wave detection units 11 and 12, the measurement can be carried out by either one of the scan in the X direction and the scan in the Y direction without using two photoacoustic wave detection units (refer to FIG. 5(b)).

The deviation in time between the time points at which the electric signals based on the photoacoustic waves obtained from the two photoacoustic wave detection units 11 and 12 rise may not change regardless of the distance of the photoacoustic wave measurement instrument 1 from the blood 2a. Even in this case, if there are three photoacoustic wave detection units, the deviation in time between the time point at which the electric signal based on the photoacoustic wave obtained from the remaining one photoacoustic wave detection unit 13 rises and the time points at which the electric signals based on the photoacoustic waves obtained from the two photoacoustic wave detection units 11 and 12 rise changes depending on the distance of the photoacoustic wave measurement instrument 1 from the blood 2a, and the position of the blood 2a can thus be measured.

This holds true for a case where the number of the photoacoustic wave detection units is odd. In other words, a deviation in time among the time points at which the electric signals based on the photoacoustic waves obtained from an even number of photoacoustic wave detection units rise may not change regardless of the distance of the photoacoustic wave measurement instrument 1 from the blood 2a. Even in this case, if there are an odd number of photoacoustic wave detection units, the deviation in time between the time point at which the electric signal based on the photoacoustic wave obtained from the remaining one photoacoustic wave detection unit rises and the time points at which the electric signals based on the photoacoustic waves obtained from the even number of photoacoustic wave detection units rise changes depending on the distance of the photoacoustic wave measurement instrument 1 from the blood 2a, and the position of the blood 2a can thus be measured.

Though a description is given of the example including the four photoacoustic wave detection units 11, 12, 13, and 14 as the second embodiment, and the example including the three photoacoustic wave detection units 11, 12, and 13 as the variation of the second embodiment, the number of the photoacoustic wave detection units may be equal to or more than five.

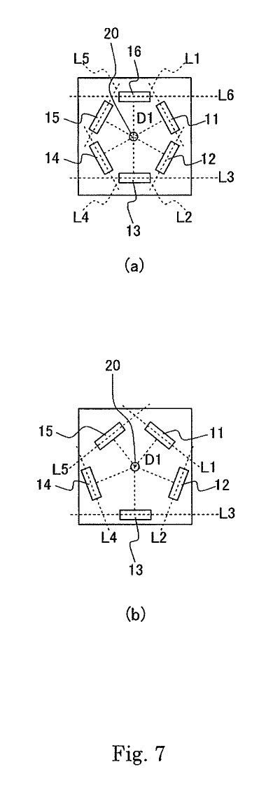

FIG. 7 includes plan views of the photoacoustic wave measurement instrument 1 according to variations of the second embodiment, and shows a case including six photoacoustic wave detection units 11, 12, 13, 14, 15, and 16 (FIG. 7(a)), and a case including five photoacoustic wave detection units 11, 12, 13, 14, and 15 (FIG. 7(b)).

Extension directions (such as L1 and L5) of at least two photoacoustic wave detection units (such as the photoacoustic wave detection units 11 and 15) out of the photoacoustic wave detection units 11, 12, 13, 14, 15, and 16 intersect with each other in the photoacoustic wave measurement instrument 1 according to the variation shown in FIG. 7(a). An extension direction L6 of the at least one photoacoustic wave detection unit 16 other than the two photoacoustic wave detection units intersects with the extension directions L1 and L5 of the two photoacoustic wave detection units 11 and 15.

It should be noted that the light output unit 20 passes through an inside of a polygon (a regular hexagon in FIG. 7(a)) constructed by straight lines extending in the extension directions L1, L2, L3, L4, L5, and L6 in the variation shown in FIG. 7(a). Moreover, all distances between the light output unit 20 and the photoacoustic wave detection units 11, 12, 13, 14, 15, and 16 are equal to D1.

Extension directions (such as L2 and L4) of at least two photoacoustic wave detection units (such as the photoacoustic wave detection units 12 and 14) out of the photoacoustic wave detection units 11, 12, 13, 14, and 15 intersect with each other in the photoacoustic wave measurement instrument 1 according to the variation shown in FIG. 7(b). An extension direction L3 of the at least one photoacoustic wave detection unit 13 other than the two photoacoustic wave detection units intersects with the extension directions L2 and L4 of the two photoacoustic wave detection units 12 and 14.

It should be noted that the light output unit 20 passes through an inside of a polygon (a regular pentagon in FIG. 7(b)) constructed by straight lines extending in the extension directions L1, L2, L3, L4, and L5 in the variation shown in FIG. 7(b). Moreover, all distances between the light output unit 20 and the photoacoustic wave detection units 11, 12, 13, 14, and 15 are equal to D1.

### Third Embodiment

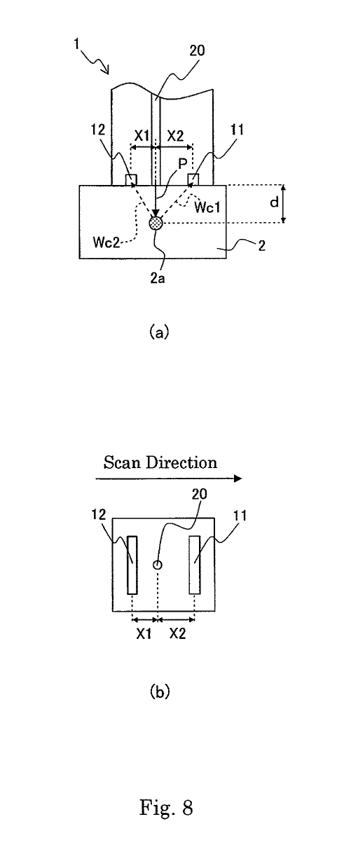

A third embodiment is different from the first embodiment in such a point that the distance between the photoacoustic wave detection unit 11 and the optical fiber 20 and the distance between the photoacoustic wave detection unit 12 and the optical fiber 20 are different from each other (refer to FIG. 8(b)).

FIG. 8 includes a cross sectional view (FIG. 8(a)) and a plan view (FIG. 8(b)) of the photoacoustic wave measurement instrument 1 according to the third embodiment of the present invention. The photoacoustic wave measurement instrument 1 includes the photoacoustic wave detection units 11 and 12, and the optical fiber (light output unit) 20. Hereinafter, like components are denoted by like numerals as of the first embodiment of the photoacoustic wave measurement instrument 1, and will be described in no more details.

The optical fiber (light output unit) 20 is the same as that of the first embodiment, and a description thereof, therefore, is omitted. The photoacoustic wave detection units 11 and 12 are also the same as those of the first embodiment. It should be noted that the positions of the photoacoustic wave detection units 11 and 12 are different from those of the first embodiment.

In other words, the photoacoustic wave detection unit 11 is separated from the optical fiber 20 in the scanning direction by a distance X2. The photoacoustic wave detection unit 12 is separated from the optical fiber 20 in the scanning direction by a distance X1. It should be noted that X1 and X2 are different from each other.

FIG. 8(a) shows such a state that the optical fiber 20 of the photoacoustic wave measurement instrument 1 is directly above the blood 2a. Reference numeral d denotes the depth of the blood 2a with respect to the surface of the measurement object 2.

The distance from the blood 2a to the photoacoustic wave detection unit 11 is the square root of d²+X2². The distance from the blood 2a to the photoacoustic wave detection unit 12 is the square root of d²+X1². Then, a deviation Δt0 between a time taken by the photoacoustic wave Wc1 to reach the photoacoustic wave detection unit 11 from the blood 2a and a time taken by the photoacoustic wave Wc2 to reach the photoacoustic wave detection unit 12 from the blood 2a is represented as (square root of ((d²+X2²)-square root of (d²+X1²))/Vs) where the velocity of the photoacoustic wave in the measurement object 2 is Vs. If Δt0 is obtained according to the above-mentioned equation, the depth d of the blood 2a has a deviation to a certain degree, and it is thus conceivable to use an approximate representative value. Moreover, if X1 and X2 are fairly larger than d, it is conceivable to neglect d, and to consider (X2−X1)/Vs as Δt0.

The deviation between the time taken by the photoacoustic wave Wc1 to reach the photoacoustic wave detection unit 11 from the blood 2a and the time taken by the photoacoustic wave Wc2 to reach the photoacoustic wave detection unit 12 from the blood 2a appears as a deviation in time of the electric signals respectively output from the photoacoustic wave detection units 11 and 12.

In other words, Δt0 is a deviation in time between the electric signals respectively output from the photoacoustic wave detection units 11 and 12 if it is assumed that the blood 2a (photoacoustic wave generation part) exists on the extension line of the optical fiber 20.

The photoacoustic wave measurement device 40 according to the third embodiment of the present invention includes the electric signal measurement units 41 and 42, the magnitude determination unit 44, the time deviation determination unit 46, and the position measurement unit 48. The configuration of the photoacoustic wave measurement device 40 according to the third embodiment of the present invention is the same as that of the first embodiment (refer to FIG. 3), and hence description thereof is omitted. Hereinafter, like components are denoted by like numerals as of the first embodiment of the photoacoustic wave measurement device 40, and will be described in no more details.

The electric signal measurement units 41 and 42 and the magnitude determination unit 44 are the same as those of the first embodiment, and a description thereof, therefore, is omitted,

The time deviation determination unit 46 determines whether the deviation in time between the electric signals output from the respective photoacoustic wave detection units 11 and 12 is in a predetermined range (for example equal to more than (Δt0−Δt) and equal to or less than (Δt0+Δt) where the predetermined range is Δt) or not based on the measurement results received from the electric signal measurement units 41 and 42. Δt0 is in the predetermined range. In other words, the predetermined range includes Δt0. A relationship Δt0−Δt>0 may hold true. In other words, the predetermined range may not include 0.

For example, the time deviation determination unit 46 determines whether a deviation in time between the rising points of the electric signals respectively output from the photoacoustic wave detection units 11 and 12 is equal to or more than (Δt0−Δt) and equal to or less than (Δt0+Δt), (or more than (Δt0−Δt) and less than (Δt0+Δt)) or not.

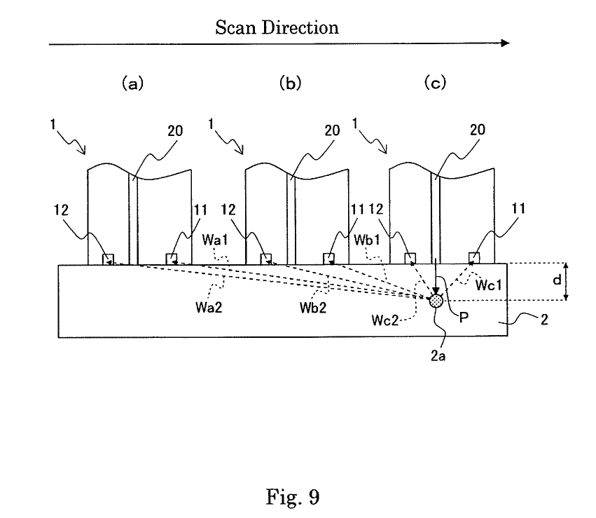

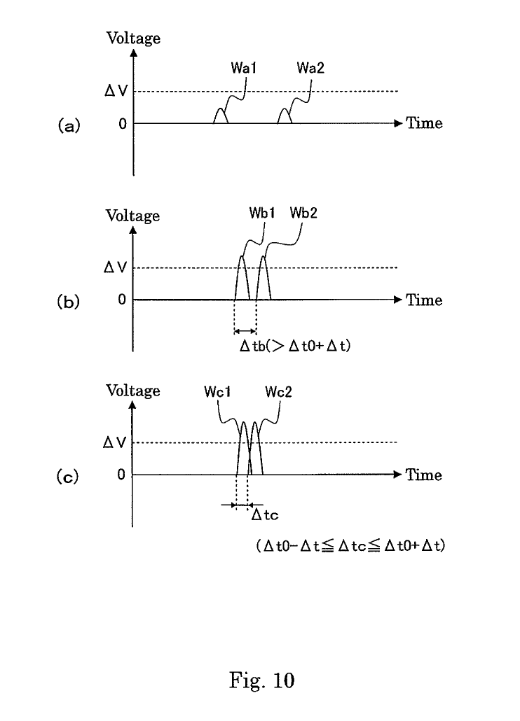

On this occasion, if the time deviation determination unit 46 determines that the deviation Δtc in time between the rising time points of the electric signals output from the respective photoacoustic wave detection units 11 and 12 is equal to or more than (Δt0−Δt), and equal to or less than (Δt0+Δt) (refer to FIG. 10(c)), the time deviation determination unit 46 outputs such a determination result (refer to FIG. 9(c)) that the optical fiber 20 of the photoacoustic wave measurement instrument 1 is directly above the blood 2a to the position measurement unit 48. If the optical fiber 20 of the photoacoustic wave measurement instrument 1 is positioned directly above the blood 2a, though a relationship Δt=Δt0 ideally holds true, if a relationship Δt0−Δt≦Δtc≦Δt0+Δt holds true considering a measurement error and a variation in depth d of the blood 2a, it is assumed to make such a determination that the optical fiber 20 of the photoacoustic wave measurement instrument 1 is directly above the blood 2a.

On the other hand, if the time deviation determination unit 46 determines that the deviation Δtb in time between the rising time points of the electric signals output from the respective photoacoustic wave detection units 11 and 12 is less than (Δt0−Δt) or more than (Δt0+Δt) (refer to FIG. 10(b)), the time deviation determination unit 46 outputs none to the position measurement unit 48. In this case, the time deviation determination unit 46 may output such a determination result that the photoacoustic wave measurement instrument 1 is positioned slightly far from the blood 2a (refer to FIG. 9(b)).

The situation where the time deviation determination unit 46 provides the position measurement unit 48 with such the determination result that the optical fiber 20 of the photoacoustic wave measurement instrument 1 is directly above the blood 2a (refer to FIG. 9(c)) means a situation where the magnitude determination unit 44 determines that the magnitudes of the electric signals respectively output from the photoacoustic wave detection units 11 and 12 are more than the threshold ΔV, and simultaneously, the time deviation determination unit 46 determines that the deviation in time between the electric signals respectively output from the photoacoustic wave detection units 11 and 12 is in the predetermined range (equal to or more than (Δt0−Δt), and equal to or less than (Δt0+Δt)).

If the position measurement unit 48 receives such the determination result that the optical fiber 20 of the photoacoustic wave measurement instrument 1 is directly above the blood 2a (refer to FIG. 9(c)) from the time deviation determination unit 46, the position measurement unit 48 measures the position of the blood 2a (photoacoustic wave generation part) at which the photoacoustic waves Wc1 and Wc2 are generated in the measurement object 2.

In this case, the position measurement unit 48 measures the position of the blood 2a (photoacoustic wave generation part) while it is assumed that the blood 2a (photoacoustic wave generation part) exists on the extension line of (directly below, for example) the optical fiber (light output unit) 20. The position measurement unit 48 receives the measurement results from the electric signal measurement units 41 and 42, thereby measuring the position of the blood 2a (photoacoustic wave generation part). For example, the depth d of the blood 2a with respect to the surface of the measurement object 2 may be measured. It is assumed that the time taken by the photoacoustic wave Wc1 (Wc2) to reach the photoacoustic wave detection unit 11 (12) from the blood 2a is T1 (T2) based on the measurement result received from the electric signal measurement unit 41 (42). Then, (T1×Vs)²=d²+X2² ((T2×Vs)²=d²+X1²) holds true, where Vs is the velocity of the photoacoustic wave in the measurement object 2. X2 (X1) and Vs are known, and the depth d of the blood 2a can thus be obtained.

A description will now be given of an operation of the third embodiment of the present invention.

FIG. 9 is a cross sectional view of the photoacoustic wave measurement instrument 1 while the photoacoustic wave measurement instrument 1 according to the third embodiment of the present invention is scanned along the measurement object 2.

When the scan of the photoacoustic wave measurement instrument 1 starts, the photoacoustic wave measurement instrument 1 is positioned far from the blood 2a as shown in FIG. 9(a).

On this occasion, the external pulse light source (not shown) generates the pulse light P, and the pulse light P is output from the optical fiber 20. The pulse light P is fed to the measurement object 2.

The pulse light P reaches the blood 2a in the blood vessel of the measurement object 2. Then, the blood 2a in the blood vessel absorbs the pulse light P, and the dilatational waves (photoacoustic waves Wa1 and Wa2) are output from the blood 2a in the blood vessel.

The photoacoustic waves Wa1 and Wa2 transmit through the measurement object 2, and reach the photoacoustic wave detection units 11 and 12. The photoacoustic wave detection units 11 and 12 convert pressures of the photoacoustic waves Wa1 and Wa2 into the electric signals (such as voltages). The voltages are fed to the electric signal measurement units 41 and 42 of the photoacoustic wave measurement device 40.

FIG. 10 includes charts showing relationships between the time and the voltage, which are the measurement results by the electric signal measurement units 41 and 42 of the photoacoustic wave measurement device 40 according to the third embodiment, and shows the measurement result of the electric signals obtained from the photoacoustic wave measurement instrument 1 in FIG. 9(a) (FIG. 10(a)), the measurement result of the electric signals obtained from the photoacoustic wave measurement instrument 1 in FIG. 9(b) (FIG. 10(b)), and the measurement result of the electric signals obtained from the photoacoustic wave measurement instrument 1 in FIG. 9(c) (FIG. 10(c)).

(a) When Photoacoustic Wave Measurement Instrument 1 is Far from Blood 2a

As shown in FIG. 9(a), the photoacoustic wave measurement instrument 1 is far from the blood 2a. Thus, the photoacoustic waves Wa1 and Wa2 are weak, and the magnitudes of the electric signals (voltages) obtained from the photoacoustic waves Wa1 and Wa2 are small, and are both equal to or less than the magnitude threshold ΔV (refer to FIG. 10(a)).

In this case, the magnitude determination unit 44 does not feed the measurement results received from the electric signal measurement units 41 and 42 to the time deviation determination unit 46. The magnitude determination unit 44 outputs such the determination result that the photoacoustic wave measurement instrument 1 is positioned far from the blood 2a (refer to FIG. 9(a)).

(b) When Photoacoustic Wave Measurement Instrument 1 is Slightly Far from Blood 2a

When the photoacoustic wave measurement instrument 1 is scanned from the state shown in FIG. 9(a), the photoacoustic wave measurement instrument 1 is positioned slightly far from the blood 2a as shown in FIG. 9(b).

As shown in FIG. 9(b), though the photoacoustic wave measurement instrument 1 is slightly far from the blood 2a, the photoacoustic wave measurement instrument 1 is closer to the blood 2a than the photoacoustic wave measurement instrument 1 in the state shown in FIG. 9(a). Thus, the photoacoustic waves Wb1 and Wb2 are stronger than the photoacoustic waves Wa1 and Wa2, and both the magnitudes of the electric signals (voltages) obtained from the photoacoustic waves Wb1 and Wb2 are more than the magnitude threshold ΔV (refer to FIG. 10(b)).

In this case, the magnitude determination unit 44 feeds the measurement results received from the electric signal measurement units 41 and 42 to the time deviation determination unit 46.

As shown in FIG. 10(b), the photoacoustic wave measurement instrument 1 is slightly far from the blood 2a, the difference between a distance traveled by the photoacoustic wave Wb1 and a distance traveled the photoacoustic wave Wb2 is not negligible. Thus, a difference between the time taken by the photoacoustic wave Wb1 to reach the photoacoustic wave detection unit 11 and the time taken by the photoacoustic wave Wb2 to reach the photoacoustic wave detection unit 12 is not negligible either. Therefore, the deviation Δtb in time between the rising time points of the electric signals obtained from the photoacoustic waves Wb1 and Wb2 is not negligible (for example, is more than the (Δt0+Δt)) referring to FIG. 4(b).

In this case, the time deviation determination unit 46 does not specifically output anything to the position measurement unit 48. The time deviation determination unit 46 outputs such the determination result that the photoacoustic wave measurement instrument 1 is positioned slightly far from the blood 2a (refer to FIG. 9(b)).

### (c) When Optical Fiber 20 of Photoacoustic Wave Measurement Instrument 1 is Directly Above Blood 2a

When the photoacoustic wave measurement instrument 1 is scanned from the state shown in FIG. 9(b), the optical fiber 20 of the photoacoustic wave measurement instrument 1 is positioned directly above the blood 2a as shown in FIG. 9(c).

As shown in FIG. 9(c), the photoacoustic wave measurement instrument 1 is closer to the blood 2a than the photoacoustic wave measurement instrument 1 in the state shown in FIG. 9(a). Thus, the photoacoustic waves Wc1 and Wc2 are stronger than the photoacoustic waves Wa1 and Wa2, and both the magnitudes of the electric signals (voltages) obtained from the photoacoustic waves Wc1 and Wc2 are more than the magnitude threshold ΔV (refer to FIG. 10(c)).

In this case, the magnitude determination unit 44 feeds the measurement results received from the electric signal measurement units 41 and 42 to the time deviation determination unit 46.

As shown in FIG. 9(c), the optical fiber 20 of the photoacoustic wave measurement instrument 1 is directly above the blood 2a. On this occasion, the distance between the photoacoustic wave detection unit 11 and the optical fiber 20 is X2, the distance between the photoacoustic wave detection unit 12 and the optical fiber 20 is X1, and X1 and X2 are different from each other referring to FIG. 8. Thus, the distance traveled by the photoacoustic wave Wc1 and the distance traveled by the photoacoustic wave Wc2 are different from each other. Thus, the time taken by the photoacoustic wave Wc1 to reach the photoacoustic wave detection unit 11 and the time taken by the photoacoustic wave Wc2 to reach the photoacoustic wave detection unit 12 are different from each other. The deviation in time between them is Δt0 as described before.

Therefore, the deviation Δtc in time between the rising time points of the electric signals obtained from the photoacoustic waves Wc1 and Wc2 is approximately equal to Δt0 (for example, Δt0−Δt≦Δtc≦Δt0+Δt) referring to FIG. 10(c).

In this case, the time deviation determination unit 46 outputs such the determination result that the optical fiber 20 of the photoacoustic wave measurement instrument 1 is positioned directly above the blood 2a (refer to FIG. 10(c)) to the position measurement unit 48. The position measurement unit 48 measures the position of the blood 2a (photoacoustic wave generation part) while it is assumed that the blood 2a (photoacoustic wave generation part) exists on the extension line of (directly below, for example) the optical fiber 20. The position measurement unit 48 receives the measurement results from the electric signal measurement units 41 and 42, thereby measuring the position (such as the depth d of the blood 2a) of the blood 2a (photoacoustic wave generation part).

It is possible to determine whether the blood 2a (photoacoustic wave generation part) exists on the extension line of (directly below, for example) the optical fiber 20 of the photoacoustic wave measurement instrument 1 (refer to FIG. 10(c)) or not (refer to FIGS. 7(a) and (b)) according to the third embodiment.

Moreover, the position measurement unit 48 measures the position of the blood 2a while it is assumed that the blood 2a exists on the extension line of the optical fiber 20 of the photoacoustic wave measurement instrument 1 in the photoacoustic wave measurement device 40. On this occasion, the photoacoustic wave measurement device 40 carries out this measurement when the blood 2a actually exists on the extension line of the optical fiber 20 of the photoacoustic wave measurement instrument 1 (refer to FIG. 9(c)). The position of the blood 2a thus can be precisely measured by the photoacoustic wave measurement instrument 1.

### Fourth Embodiment

The photoacoustic wave measurement instrument 1 according to a fourth embodiment is different from the photoacoustic wave measurement instrument 1 according to the third embodiment in that the photoacoustic wave measurement instrument 1 according to the fourth embodiment includes at least three photoacoustic wave detection units.

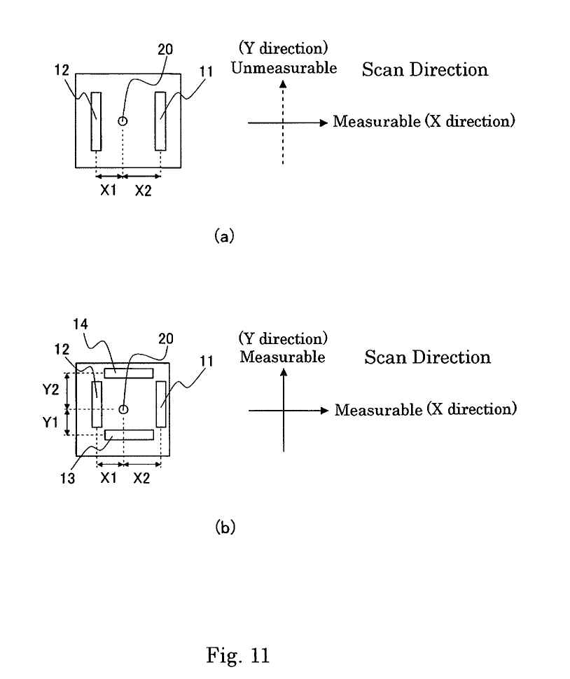

FIG. 11 includes a plan view (FIG. 11(a)) of the photoacoustic wave measurement instrument 1 according to the third embodiment of the present invention and a plan view (FIG. 11(b)) of the photoacoustic wave measurement instrument 1 according to the fourth embodiment of the present invention.

The photoacoustic wave measurement instrument 1 according to the fourth embodiment of the present invention includes photoacoustic wave detection units 11, 12, 13, and 14, and the optical fiber (light output unit) 20. In the following section, like components are denoted by like numerals as of the third embodiment, and will be explained in no more details.

Referring to FIG. 11(b), the photoacoustic wave measurement instrument 1 according to the fourth embodiment is in contact with the measurement object 2, and scans the measurement object 2 thereabove, for example from the left to the light (in the X direction) or scans from the rear to the front (in the Y direction). In other words, the scanning can be carried out in the two directions orthogonally intersecting with each other. It should be noted that the scan can be carried out at the same time in the two directions orthogonal to each other (for example, from the obliquely left rear to the obliquely right front).

The photoacoustic wave detection units 11 and 12, and the optical fiber (light output unit) 20 are the same as those of the third embodiment, and a description thereof, therefore, is omitted.

It should be noted that, referring to FIG. 11(b), the photoacoustic wave measurement instrument 1 according to the fourth embodiment has four photoacoustic wave detection units.

Configurations of the photoacoustic wave detection unit 13 and 14 are the same as the configurations of the photoacoustic wave detection units 11 and 12. It should be noted that the photoacoustic wave detection units 13 and 14 are separated respectively by distances Y1 and Y2 in the Y direction from the optical fiber 20. It should be noted that Y1 and Y2 are different from each other. Any of X1, X2, Y1, and Y2 may be known values different from one another. In this case, the photoacoustic wave detection units 11, 12, 13, and 14 are arranged at the known distances X2, X1, Y1, and Y2 from the optical fiber 20, which are different from one another.

It should be noted that the photoacoustic wave detection units 11 and 12 extend in the direction (Y direction) orthogonal to the direction of the scan (X direction). Moreover, it should be noted that the photoacoustic wave detection units 13 and 14 extend in the direction (X direction) orthogonal to the direction of the scan (Y direction). Thus, the extension direction (Y direction) of the photoacoustic wave detection unit 11 and the extension direction (Y direction) of the photoacoustic wave detection unit 12 are parallel with each other, and the extension direction (X direction) of the photoacoustic wave detection unit 13 and the extension direction (X direction) of the photoacoustic wave detection unit 14 intersect with the extension direction (Y direction) of the photoacoustic wave detection unit 11 and the extension direction (Y direction) of the photoacoustic wave detection unit 12.

The configuration of the photoacoustic wave measurement device 40 according to the fourth embodiment of the present invention is the same as that of the third embodiment, and hence an illustration and a description thereof is therefore omitted. It should be noted that the electric signal measurement units are provided as many as the number (four) of the photoacoustic wave detection units.

A description will now be given of an operation of the fourth embodiment of the present invention while comparing the fourth embodiment with the third embodiment.

The photoacoustic wave measurement instrument 1 according to the third embodiment of the present invention shown in FIG. 11(a) can measure the measurement object 2 while the scan is carried out in the X direction so as to pass directly above the blood 2a as described before.

However, if the scan is carried out in the Y direction, the measurement object 2 may not be measured. Even if the optical fiber 20 of the photoacoustic wave measurement instrument 1 is not directly above the blood 2a of the photoacoustic wave measurement instrument 1, a deviation in time between the time point at which the electric signal obtained from the photoacoustic wave received by the photoacoustic wave detection unit 11 rises and the time point at which the electric signal obtained from the photoacoustic wave received by the photoacoustic wave detection unit 12 rises can become Δt0.

However, the photoacoustic wave measurement instrument 1 according to the fourth embodiment of the present invention shown in FIG. 11(b) can measure the measurement object 2 even if the scan in the Y direction is carried out. It is only necessary to determine whether or not a deviation Δtc′ in time between the time point at which the electric signal obtained from the photoacoustic wave received by the photoacoustic wave detection unit 13 rises and the time point at which the electric signal obtained from the photoacoustic wave received by the photoacoustic wave detection unit 14 rises is approximately equal to a predetermine value Δt0′ (=((square root of d²+Y2²)·(square root of d²+Y1²))/Vs). For example, it is only necessary to determine whether Δt0′−Δt≦Δtc′≦Δt0′+Δt holds true or not.

The distance traveled by the photoacoustic wave received by the photoacoustic wave detection unit 11 and the distance traveled by the photoacoustic wave received by the photoacoustic wave detection unit 12 are different from each other depending on the distance of the photoacoustic wave measurement instrument 1 from the blood 2a (refer to the third embodiment), and the deviation in time between the time points at which the electric signals obtained from the respective photoacoustic waves rise thus changes. Therefore, the photoacoustic wave measurement instrument 1 according to the fourth embodiment of the present invention shown in FIG. 11(b) can measure the measurement object 2 while the scan is carried out in the X direction so as to pass directly above the blood 2a.

The photoacoustic wave measurement apparatus 40 according to the fourth embodiment converts the photoacoustic waves obtained from the photoacoustic wave detection units 11, 12, 13, and 14 into the electric signals by the four electric signal measurement units provided as many as the number of the photoacoustic wave detection units. Then, the magnitude determination unit 44 determines whether each of the magnitudes (voltages) of the electric signals is more than the magnitude threshold ΔV or not. Then, the time deviation determination unit 46 determines whether the deviation Δtc in time between the rise time points of electric signals based on the photoacoustic waves obtained from the photoacoustic wave detection units 11 and 12 is approximately equal to Δt0 or not, and determines whether the deviation Δtc′ in time between the rise time points of electric signals based on the photoacoustic waves obtained from the photoacoustic wave detection units 13 and 14 is approximately equal to Δt0′ or not. As a result, the measurement object 2 can be measured.

The photoacoustic wave measurement instrument 1 according to the fourth embodiment can carry out the scan above the measurement object 2 in the two directions orthogonal to each other, for example, from the left to the right (in the X direction), or from the rear to the front (in the Y direction). It should be noted that the scan can be carried out at the same time in the two directions orthogonal to each other (for example, from the obliquely left rear to the obliquely right front).

It should be noted that variations of the photoacoustic wave measurement instrument 1 according to the fourth embodiment of the present invention are conceivable.

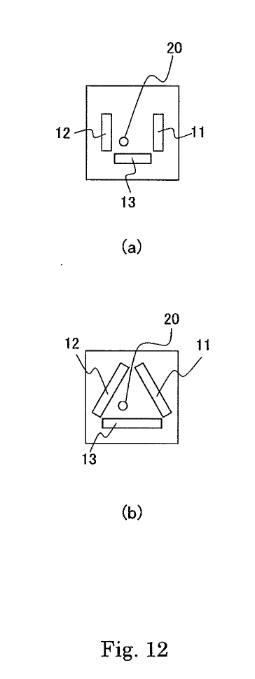

FIG. 12 includes plan views of the photoacoustic wave measurement instrument 1 according to a variation of the fourth embodiment, and shows a case where the photoacoustic wave detection units 11 and 12 are parallel with each other (FIG. 12(a)) and a case where the photoacoustic wave detection units 11 and 12 intersect with each other (FIG. 12(b)).

The photoacoustic wave measurement instrument 1 includes the three photoacoustic wave detection units 11, 12, and 13 in the variation of the fourth embodiment shown in FIG. 12.

FIG. 12(a) is the plan view of the photoacoustic wave measurement instrument 1 according to the variation where the photoacoustic wave detection units 11 and 12 are parallel with each other. The variation shown in FIG. 12(a) is in a shape where the photoacoustic wave detection unit 14 is absent in the photoacoustic wave measurement instrument 1 (refer to FIG. 11(b)) according to the fourth embodiment. Moreover, the variation shown in FIG. 12(a) corresponds to the variation of the second embodiment shown in FIG. 6(a) where the distances between the light output unit 20 and the respective photoacoustic wave detection units 11, 12, and 13 are known values different from one another.

FIG. 12(b) is the plan view of the photoacoustic wave measurement instrument 1 according to the variation where the photoacoustic wave detection units 11 and 12 intersect with each other. Moreover, the variation shown in FIG. 12(b) corresponds to the variation according to the second embodiment shown in FIG. 6(b) where the distances between the light output unit 20 and the respective photoacoustic wave detection units 11, 12, and 13 are known values different from one another.

In FIG. 12, the measurement object 2 can be measured by using the deviation in time between the rise time points of the electric signals based on the photoacoustic waves obtained from the photoacoustic wave detection units 11 and 12, and the deviation in time between the rise time points of the electric signals based on the photoacoustic waves obtained from the photoacoustic wave detection units 11 (or 12) and 13.

It should be noted that the photoacoustic wave detection units 11 and 12 are parallel with each other (refer to FIG. 12(a)) or intersect with each other (refer to FIG. 12(b)) in the variation of the fourth embodiment, and the photoacoustic wave detection unit 12 is never disposed on the extension of the photoacoustic wave detection unit 11.

Whether the photoacoustic wave detection units 11 and 12 are parallel with each other, or intersect with each other as shown in FIG. 12, if there is the one photoacoustic wave detection unit 13 extending so as to intersect with the extension directions of the photoacoustic wave detection units 11 and 12, the measurement can be carried out by either one of the scan in the X direction and the scan in the Y direction without two photoacoustic wave detection units (refer to FIG. 12(b)).

There is a case where the position of the blood 2a cannot be measured only by the deviation in time between the rise time points of the electric signals based on the photoacoustic waves obtained from the two photoacoustic wave detection units 11 and 12. Even in this case, if there are three photoacoustic wave detection units, the deviation in time between the time point at which the electric signal based on the photoacoustic wave obtained from the remaining one photoacoustic wave detection unit 13 rises and the time point at which the electric signal based on the photoacoustic wave obtained from the photoacoustic wave detection unit 11 (or 12) rises can be further used to measure the position of the blood 2a. This holds true for a case where the number of the photoacoustic wave detection units is odd.

Though a description is given of the example including the four photoacoustic wave detection units 11, 12, 13, and 14 as the fourth embodiment, and the example including the three photoacoustic wave detection units 11, 12, and 13 as the variation of the fourth embodiment, the number of the photoacoustic wave detection units may be equal to or more than five.

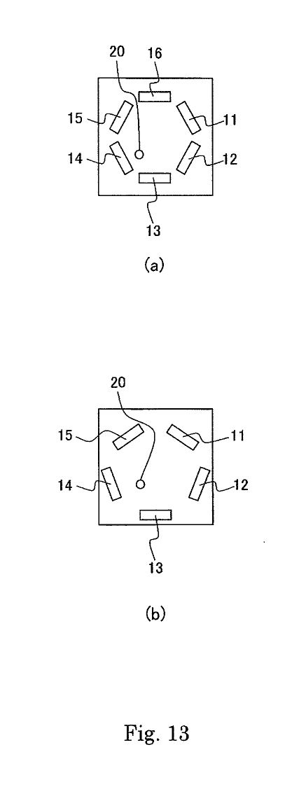

FIG. 13 includes plan views of the photoacoustic wave measurement instrument 1 according to variations of the fourth embodiment, and shows a case including six photoacoustic wave detection units 11, 12, 13, 14, 15, and 16 (FIG. 13(a)), and a case including five photoacoustic wave detection units 11, 12, 13, 14, and 15 (FIG. 13(b)).

The variation shown in FIG. 13(a) corresponds to the variation according to the second embodiment shown in FIG. 7(a) where the distances between the light output unit 20 and the respective photoacoustic wave detection units 11, 12, 13, 14, 15, and 16 are known values different from one another.

The variation shown in FIG. 13(b) corresponds to the variation according to the second embodiment shown in FIG. 7(b) where the distances between the light output unit 20 and the respective photoacoustic wave detection units 11, 12, 13, 14, and 15 are known values different from one another.

Moreover, the above-described embodiment may be realized in the following manner. A computer is provided with a CPU, a hard disk, and a media (such as a floppy disk (registered trade mark) and a CD-ROM) reader, and the media reader is caused to read a medium recording a program realizing the above-described respective components such as the photoacoustic wave measurement device 40, thereby installing the program on the hard disk. This method may also realize the above-described functions.

What is claimed is:

1. A photoacoustic wave measurement instrument comprising:
a light output unit that outputs light; and
at least three photoacoustic wave detection units that respectively receive photoacoustic waves generated by the light in a measurement object, and convert the photoacoustic wave into electric signals, wherein:
at least two of the photoacoustic wave detection units have extension directions parallel with or intersecting with each other; and
at least one of the photoacoustic wave detection units other than the at least two photoacoustic wave detection units has an extension direction intersecting with the extension directions of the at least two photoacoustic wave detection units.

2. The photoacoustic wave measurement instrument according to claim 1, wherein the light output unit passes inside a polygon constructed by straight lines extending in the extension directions of the photoacoustic wave detection units.

3. The photoacoustic wave measurement instrument according to claim 1, wherein the number of photoacoustic wave detection units is odd.

4. The photoacoustic wave measurement instrument according to claim 1, wherein the photoacoustic wave detection units are arranged at an equal distance from the light output unit.

5. The photoacoustic wave measurement instrument according to claim 1, wherein the photoacoustic wave detection units are arranged at known different distances from the light output unit.

6. The photoacoustic wave measurement instrument according to claim 2, wherein the number of photoacoustic wave detection units is odd.

7. The photoacoustic wave measurement instrument according to claim 2, wherein the photoacoustic wave detection units are arranged at an equal distance from the light output unit.

8. The photoacoustic wave measurement instrument according to claim 2, wherein the photoacoustic wave detection units are arranged at known different distances from the light output unit.
